(19) United States
(12) Patent Application Publication
(10) Pub. No.: US 2021/0338356 A1
(43) Pub. Date: Nov. 4, 2021

(54) Method and Apparatus for Securing Surgical Instruments During Surgery

(71) Applicant: William T Bresnihan, Jr., Sunrise, FL (US)

(72) Inventor: William T Bresnihan, Jr., Sunrise, FL (US)

(21) Appl. No.: 16/864,398

(22) Filed: May 1, 2020

(51) Int. Cl. A61B 46/23

(52) U.S. Cl. CPC A61B 46/23; A61B 2046/205

(57) ABSTRACT

A method and apparatus for detachably securing surgical instruments to a sterile field during surgery, comprising a sterile mat disposed on a specified zone of a sterile surgical drape which mat includes a surface having one constituent of a touch fastener, a plurality of surgical instruments each having secured thereto the opposing constituent of a touch fastener where, when one of the plurality of surgical instruments is dropped onto the mat it will be releasably held to the mat.

The present invention relates generally to the field of medical surgery and more particularly to a method and apparatus for effortlessly securing unused surgical instruments during surgery.

### BACKGROUND

Surgery requires the use and quick accessibility of many different types of instruments such as electro-cautery, suction and irrigation devices and others. The prior art has recognized the need for storing surgical devices and instruments that are not in immediate use in a location close to the surgical site where they can be easily accessed and quickly stowed. Open ended pockets attached to the surgical drape have found frequent use as temporary storage for surgical instruments. Such pockets require the surgeon to return the instrument to the pocket after each use and to look briefly at the pocket when storing an instrument to avoid missing the pocket and dropping the instrument on the floor. This often results in surgeons not using the pockets as intended and disregarding their use entirely, which frequently results in sterile instruments being dropped to the floor and becoming contaminated, therefore requiring sterile replacements. Such pockets are often ineffective due to a lack of pocket adhesion to the sterile drapes, poor initial placement of the pocket on the sterile drapes and the tendency of the pockets to collapse when high suction devices such as suction/irrigation devices are inserted into the pocket, requiring two handed removal of these devices from the pocket. Improvements have been made on the fundamental pocket stowage. For example, U.S. Pat. No. 5,082,111 to Corbitt, Jr. et al discloses a surgical instrument holder having a base that can be attached to a patient's body or drape. A plurality of hinged flaps are attached on one side to the base and on a second side are releasably attached to the base by way of hook and loop fasteners. When the releasable side of the flap is opened a surgical instrument can be inserted against the base. Closing the flap by engaging the hook and loop fastener causes the instrument to be detained against the base. Both storing and releasing the instrument require attention to the holding device and both operations require the use of two hands, one to position or hold the instrument and one to operate the flap.

Accordingly, it is the object of the present invention to provide apparatus and an associated method for effortlessly securing surgical instruments to a specified zone on a surgical drape. A related objective is to provide a method for placing a surgical instrument in a temporary holding position that does not require diverting the attention of the operating surgeon and does not require the use of hands.

### BRIEF DESCRIPTION OF THE DRAWINGS

FIG. 1 is a perspective view of a surgical patient partially covered with an overlying drape to which is attached a fabric mat, having a surface comprising the first constituent of a touch fastener, on which a number of surgical instruments are disposed each having attached thereto the second constituent of a touch fastener, releasably attaching the surgical instruments to the mat.

FIG. 2 illustrates the prior art system of pockets to temporarily hold surgical instruments during a surgical procedure.

FIG. 3 is an enlarged view of the mat and the releasably attached surgical instruments as shown in FIG. 1.

FIG. 4 is a perspective view of the application of a surgical instrument being fitted with a fabric strip have loop components.

### SUMMARY OF THE INVENTION

A fabric mat having the hook components of a touch fastener is either adhered to a surgical drape or incorporated into the drape. The mat is adapted to catch and releasably hold a surgical instrument when the instrument is dropped onto the mat, the instrument having the loop components of a touch fastener adhered thereto.

### DETAILED DESCRIPTION

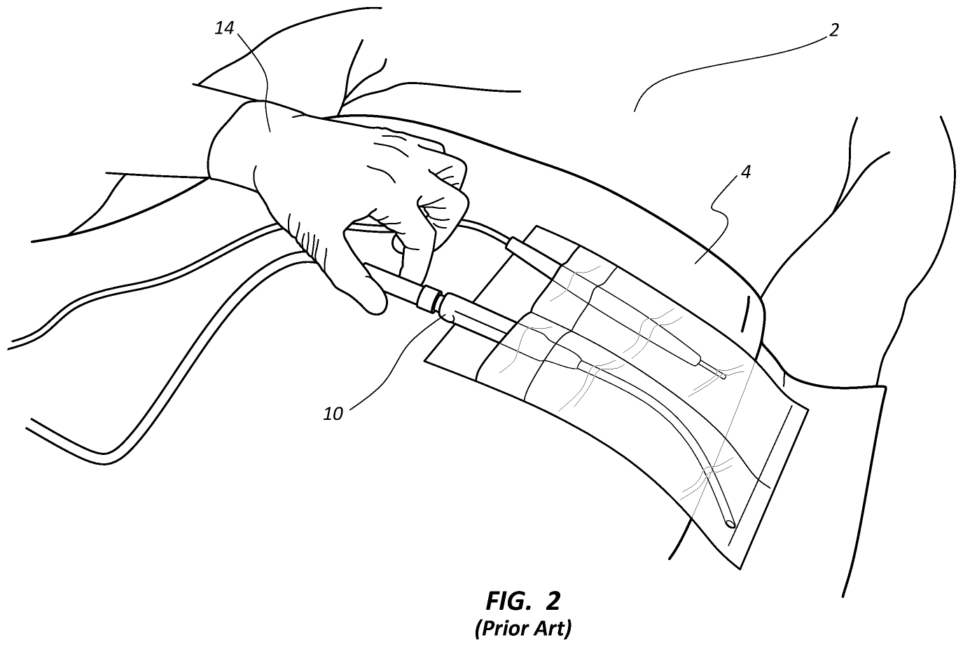

FIG. 2 illustrates the prior art open ended pockets attached to the surgical drape 4 which partially covers a patient 2. A surgeon's hand 14 is shown withdrawing a surgical instrument 10 from one of the pockets.

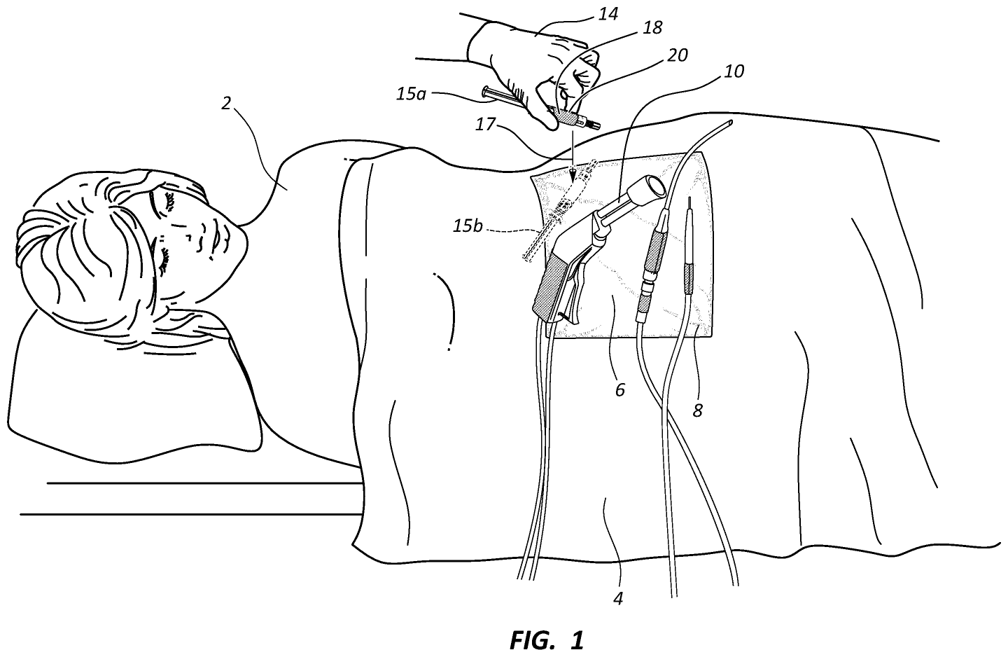

Referring to FIG. 1, a surgical patient 2 is partially covered in the bodily area of the surgery with a sterile drape 4. Adhered to the drape by traditional means, or made integrally as part of the drape, is a mat 6 having a surface comprising the hook components 8 of a touch fastener. Releasably secured to the mat, by engagement of the loop components of a touch fastener, are a variety of surgical instruments 10 each having secured thereto a fabric strip containing the loop components 20 of a touch fastener. A surgeon's hand 14 is shown holding a surgical instrument 15a having adhered thereto a strip of fabric 18 having the loop components of a touch fastener. An arrow 17 indicates that the instrument 15a is being dropped onto the mat 6. The dropped instrument 15b is shown in dotted lines in position on the surface of the mat 6, its loop components 20 having been engaged by the hook components 8 of the mat 6. After dropping the instrument on the mat the surgeon may pick up a chosen different instrument from the variety of instruments 10 by merely pulling the chosen instrument from its releasable hook and loop connection to the mat.

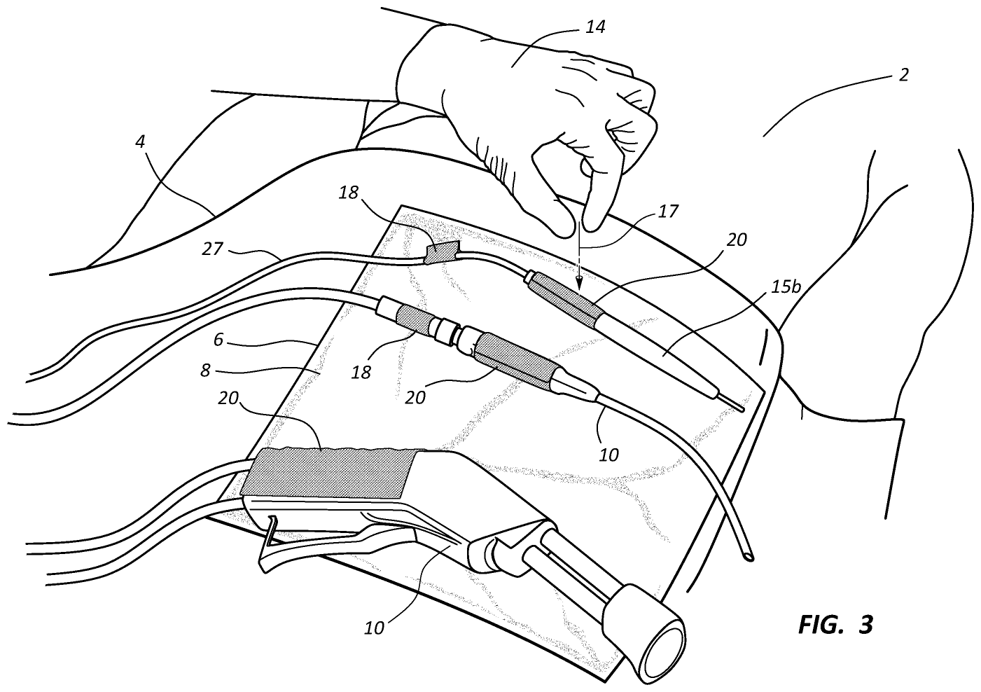

FIG. 3 is an enlarged view of the mat 6 with the surgical instruments 10 adhered thereto through the connectivity of the respective touch fasteners. The instrument 15b has been dropped onto the mat 6 by the surgeon's hand 14, as indicated by arrow 17. FIG. 3 illustrates that not only may surgical instruments be secured to the mat 6, but so can such other items as tubing or wiring 27.

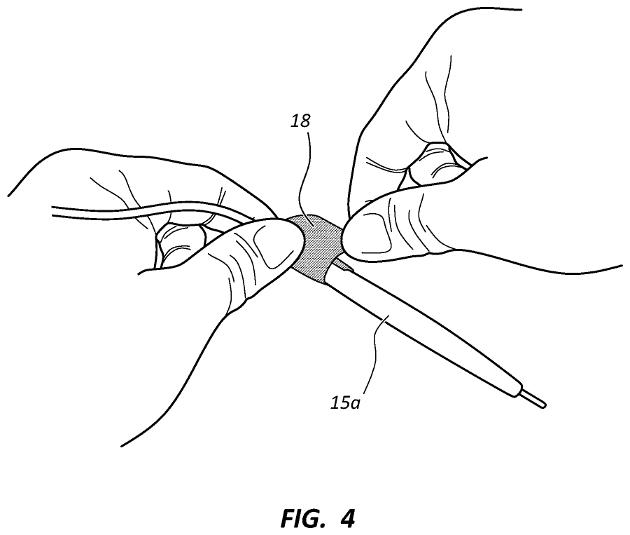

A large area fabric pad containing the loop components of a touch fastener can be cut into a number of different sized strips 18 to be applied to a surgical instrument or device 15a, as shown in FIG. 4. These adhesive loop component fabric strips, may or may not include radiopaque markers.

I claim:

1. A method of effortlessly detachably securing surgical instruments to a sterile field during surgery, comprising the steps of;
adhering a sterile mat comprising the first constituent of a touch fastener to a specified zone of a sterile surgical drape;
applying a sterile strip comprising the second constituent of a touch fastener to a surgical instrument; and
dropping the surgical instrument onto the mat.

2. The method of claim 1 where the matt comprises the hook element of a touch fastener.

3. The method of claim 2 where the strip comprises the loop element of a touch fastener.

4. The method of claim 3 where the surgical instrument includes tubing.

5. A method of effortlessly releasably securing surgical instruments to a sterile field during surgery, comprising the steps of:
adhering a sterile mat comprising the hook element of a touch fastener to a specified zone of a sterile surgical drape;
applying a sterile strip comprising the loop element of a touch fastener to a surgical instrument; and
dropping the surgical instrument onto the mat.

6. An interactive assembly for securing the position of surgical instruments dropped onto a collection zone during a surgical procedure, comprising,
a surgical drape having a zone for collecting and holding surgical instruments during a surgical procedure,
at least one matt having first and second opposing sides where the second side includes the hook constituent of a hook and loop fastener and the first side is detachably attached to the holding zone of the surgical drape,
a plurality of surgical instruments,
a plurality of material strips each having first and second opposing sides where the second side includes the loop constituent of a hook and loop fastener and the first side is detachably attached to a portion of a respective one of the plurality of surgical instruments,
where, when the respective one of the surgical instruments is dropped onto the matt, the respective one of the surgical instruments will be releasably adhered to the matt.

7. The assembly of claim 6 where the plurality of surgical instruments includes tubing.

8. The assembly of claim 6 where the plurality of surgical instruments includes electrical wiring.
